(19) United States
(12) Patent Application Publication
(10) Pub. No.: US 2022/0280219 A1
(43) Pub. Date: Sep. 8, 2022

(54) ELECTRODE ARRANGEMENT

(71) Applicant: Erbe Elektromedizin GmbH, Tuebingen (DE)

(72) Inventors: Tjark Brandt, Tuebingen (DE); Uwe Schnitzler, Tuebingen (DE)

(73) Assignee: Erbe Elektromedizin GmbH, Tuebingen (DE)

(21) Appl. No.: 17/628,727

(22) PCT Filed: Jul. 21, 2020
(86) PCT No.: PCT/EP2020/070567

§ 371 Date: Jan. 20, 2022

(30) Foreign Application Priority Data
Jul. 23, 2019 (EP) 19187863.6

(51) Int. Cl. A61B 18/04

(52) U.S. Cl. CPC A61B 18/042; A61B 2018/00589

(57) ABSTRACT

An electrode for an electrosurgical instrument for plasma coagulation. The electrode has a heat dissipation element arranged such that the thermal resistance of the electrode, measured in the longitudinal direction (in distal or proximal direction), is 2: 300 WI (m*K). The heat dissipation element may be formed by a coating having a higher electrical conductivity and a higher thermal conductivity than the material of the electrode main part.

The invention refers to an electrode arrangement for an electrosurgical instrument, particularly for an instrument for plasma coagulation of biological tissue.

Instruments for tissue coagulation are known from different documents as well as from practical experience. For this it is referred to DE 10 2011 116 678 A1 as well as DE 699 28 370 T2. Both documents disclose instruments having electrodes that are, for example, configured in a ring-shaped manner and can consist of a suitable material that is characterized by its heat resistance, such as among others also tungsten.

Further a plasma coagulation instrument having a hose-like instrument body, inside the lumen of which a hexagonal electrode consisting of metal is arranged, is known from DE 100 30 111 A1. It is connected with an electrical supply line in order to be able to supply the electrode arranged at the distal end of the hose with RF voltage. An electrical discharge origins from the tip configured at the distal end of the plate-shaped electrode, whereby a plasma stream, particularly a noble gas plasma stream, can be created. The gas stream flowing around the electrode concurrently serves to dissipate heat from the electrode, whereby excessive heating thereof shall be avoided. Due to the heat dissipation, in addition a minimization of the burn-off behavior at the discharge section of the electrode shall be achieved and in so doing, the lifetime of the instrument shall be increased.

An efficient cooling of the electrode platelet by the gas stream, however, requires a high gas flow, which is not always desired.

Furthermore, it is known from WO 2005/046495 A1 to use an ignition electrode from tungsten wire, the end of which is located at a distal end of an otherwise hose- or tube-shaped instrument body. The tungsten wire arranged in the lumen of this body is held in a certain distance to its distal end by means of a platelet on which it is attached and that serves for its cooling. However, the heat dissipation from the tungsten wire is impeded by the transition location between the platelet and the tungsten wire.

Due to electrode burn-off, particles, particularly metal particles, can get in the spark and/or the plasma stream and thus finally in living tissue, which is increasingly rejected. It is therefore object of the invention to provide a concept with which the material removal of the used electrode of an electrosurgical instrument can be reduced during use.

This object is solved by mean of the electrode arrangement according to claim 1:

The electrode arrangement according to the invention comprises an electrode on which a tip is configured that is orientated in distal direction. The electrode cross-section increases in proximal direction away from the tip. In connection with the additional feature according to which the electrode consists of a material or a material combination, the thermal conductivity of which is larger than 20 W/(m*K), a severely reduced electrode burn-off can be achieved thereby.

The electrode cross-section can increase originating from the distal tip in a stepwise manner or also continuously until the electrode touches the wall of the lumen in which it is arranged. If the cross-section increase from the tip to a proximal part of the electrode occurs in a stepwise manner, one or multiple steps can be provided for this purpose. The tip of the electrode (and the flank area of the electrode adjoining the tip) is the location from which typically a spark or a plasma stream originates. The tip and the directly adjoining part of the electrode thus forms the part of the electrode in which the discharge root point of the electrical discharge is located. At this root point a current concentration occurs that concurrently forms a heat source. Due to both measures, namely the cross-section increase of the electrode in proximal direction and the use of a material or a material combination for the electrode, the thermal conductivity of which is larger than 20 W/(m*K), the heat created at the electrode root point is largely more effectively dissipated than it has been the case up to present with the use of stainless steel electrodes having the same structural form. Therefore, the inventive electrode is characterized particularly in that the thermal conductivity thereof measured from the tip in proximal direction and/or measured transverse to the proximal direction is larger than the thermal conductivity of stainless steel. Preferably, the thermal conductivity λ of the electrode is larger than 27 W/(m*K), further preferably larger than 50 W/(m*K), larger than 100 W/(m*K), larger than 200 W/(m*K), larger than 300 W/(m*K) and particularly larger than 400 W/(m*K).

The combination of an electrode geometry in which the electrode cross-section increases away from the tip in proximal direction, whereby the electrode is made of high thermal conductive material or a high thermal conductive material combination, allows the use of a tip with particularly small radius of curvature that can be particularly smaller than 1/10 of the maximum transverse dimension of the electrode. In doing so, a high field strength can be achieved at the electrode tip that can lead to the formation of a spark and a plasma, also in case of low RF voltages and RF currents. Then the electrode is especially ignitable.

Preferably the electrode is configured in a plate-shaped manner, wherein the increase of the electrode cross-section is achieved by an increasing transverse dimension of the electrode along the axial direction in proximal direction away from the tip. The transverse dimension can increase continuously, whereby a particularly good heat dissipation is achieved. A continuous cross-section and transverse dimension increase can be achieved in that steplessly configured edges adjoin the tip of the electrode.

The electrode can be configured as platelet that comprises two flat sides that are connected with one another by means of narrow sides. Between the narrow sides and the flat sides, edges can be formed. Such an electrode can be provided as cut sheet metal, for example.

Particularly metals with high thermal conductivity, such as silver, copper, tungsten, hard metal (e.g. tungsten carbide sintered metal) or the like are suitable as electrode material. The high thermal conductivity avoids heat build-up at the tip of the electrode and facilitates discharge of heat in the entire electrode body and thus also facilitates heat dissipation to the gas stream. Thus, a relatively low gas stream is sufficient for cooling the electrode.

According to an alternative of the invention, the electrode consists of a material combination that can be made in that the electrode consists of a base body that comprises at least one surface on which a heat dissipation device is attached. The heat dissipation device thereby extends in distal direction, preferably up to the tip of the electrode, at least up to an area that is occupied by the discharge root point during operation. Thus, the heat created there can be directly transferred to the heat dissipation device without requiring heat transfer from the electrode to the heat dissipation device. In other words, the heat dissipation device is in direct contact with the heat source, here in form of the discharge root point. In proximal direction the heat dissipation device extends preferably at least up to an area of the electrode in which it comprises its maximum transverse dimension.

In the simplest case the electrode consists of a base material on which a thermally conductive overlay is applied as heat dissipation device, e.g. in form of a thermally conductive coating. This layer extends preferably up to the tip of the electrode and over large sections of the flat sides or over the entire flat sides of the electrode. The coating can also extend over the narrow sides of the electrode. For example, if the electrode is made of stainless steel or another less well heat-conducting material, the heat dissipation device is made of particularly good heat-conductive material, such as e.g. silver, carbon-like diamond (CLD) or the like. However, preferably the heat dissipation device is made of a metallic material that is also electrically conductive, such that the heat dissipation device, e.g. in form of the heat dissipating layer, contributes to the current conduction and can get into direct contact with the discharge root point. Preferably the heat dissipation device, that is e.g. configured as heat-conductive coating, is also particularly well electrically conductive. It is particularly advantageous, if the electrical conductivity of the coating is larger than the electrical conductivity of the base material.

In a particularly preferred embodiment the heat dissipation device comprises thus an electrical conductivity and particularly also a thermal conductivity that is respectively larger than the electrical and thermal conductivity than the base body.

During application of the electrode with radio frequency alternating voltage a current flow can concentrate thereby on the electrically highly conductive coating, whereby the ohmic conduction losses are small, due to the high surface conductivity. In doing so, the heat creation at the electrode due to ohmic losses is reduced. The reduction of the heat creation remarkably contributes to the increase of lifetime of the electrode and to reduction of the material removal.

In the drawings embodiments of the invention are illustrated. The drawings show:

FIG. 1 an inventive instrument, the assigned supplying apparatus and a neutral electrode in very schematic partly perspective illustration,

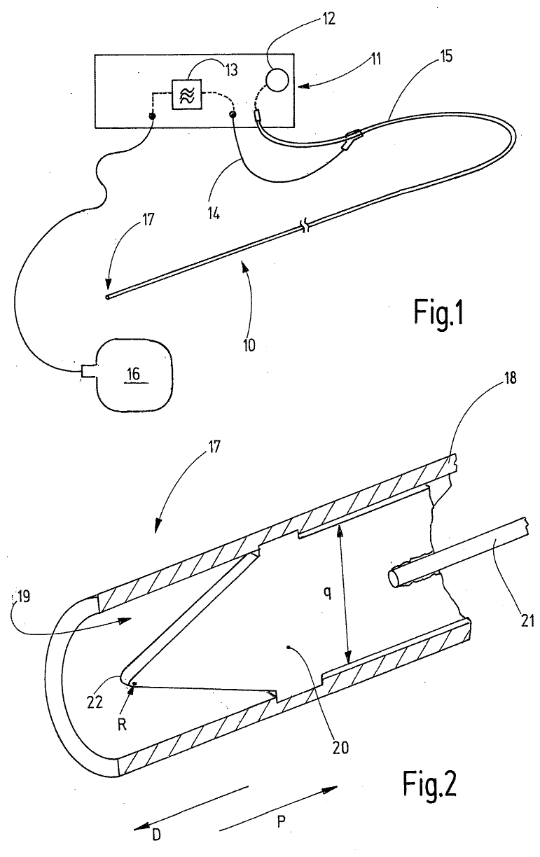

FIG. 2 the distal end of the instrument according to FIG. 1 in perspective schematic longitudinal-section illustration,

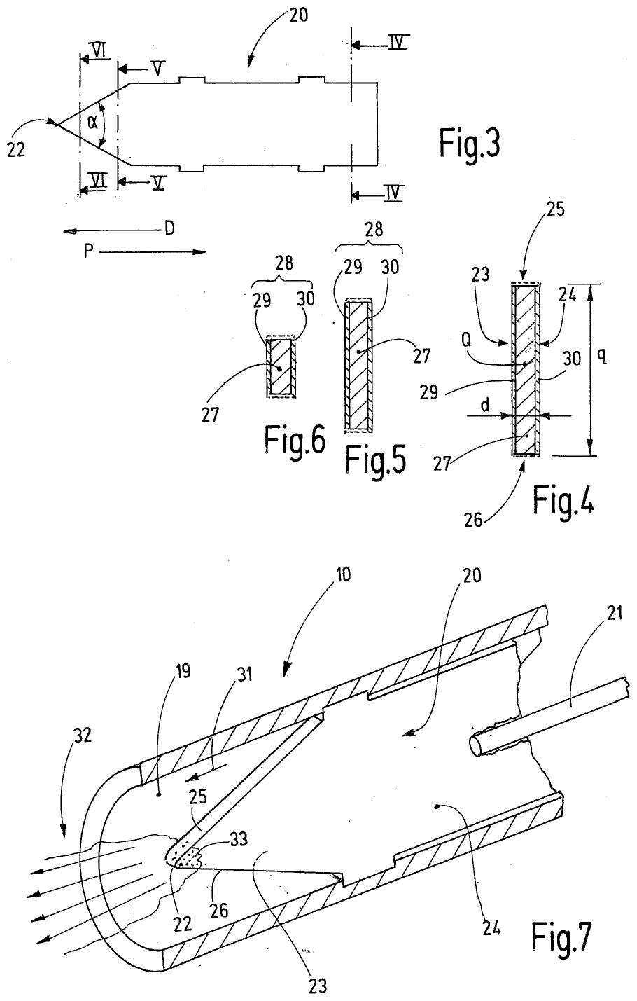

FIG. 3 the electrode of the instrument according to FIG. 2 in side view,

FIGS. 4, 5 and 6 different cross-sections of the electrode according to FIG. 3,

FIG. 7 the instrument according to FIGS. 2-6 in a sectional perspective illustration during operation,

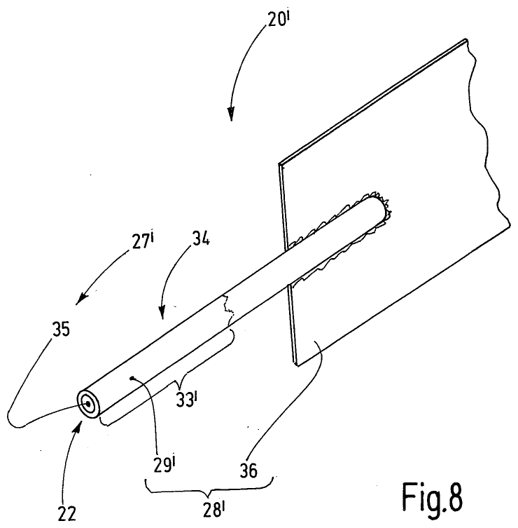

FIG. 8 a modified embodiment of an electrode for the instrument according to FIG. 2.

In FIG. 1 an instrument 10 is illustrated that serves for plasma-based tissue treatment. The tissue treatment can comprise ablation, coagulation, cutting or other types of treatment.

The instrument is connected to an apparatus 11 that contains a gas source 12, e.g. an argon source, as well as a generator 13 for electrical supply of the instrument 10. It is connected via respective connection means with a line 14 that leads to the instrument 10 and that is introduced in a line 15 via which instrument 10 is supplied with gas. The generator 13 is in addition connected via respective connection means with a neutral electrode 16 that is to be attached to a patient prior to the use of instrument 10. The following description, however, also applies for instruments with other neutral electrode configuration.

The instrument 10 comprises a distal end 17 that is separately illustrated in FIG. 2. As apparent, a tube or hose 18 is part of instrument 10 that surrounds a lumen 19 that is open at the distal end 17 of hose 18. In the area of the distal end 17 hose 18 can be provided with an inner or outer reinforcement, e.g. in form of a ceramic sleeve, which is not further illustrated in FIG. 2. The hose 18 can thus be configured with one or multiple layers. Examples for instruments with a ceramic sleeve inserted in the open end of hose 18 can be taken from WO 2005/046495 A1.

An electrode 20 is arranged in lumen 19 that is electrically connected with a wire 21 extending through lumen 19 and being part of line 14. The wire 21 can be welded to electrode 20 or can also be connected mechanically, e.g. by crimping.

Electrode 20 preferably comprises the basic shape illustrated in FIG. 3. At its distal end a sharp or at most slightly rounded tip 22 is configured on the electrode 20, the radius of curvature R of which (FIG. 2) is as small as possible and preferably smaller than one tenth of the transverse dimension q that is to be measured transverse to the axial direction and largely corresponds to the inner diameter of lumen 19. Electrode 20 is preferably configured in a plate-shaped manner, i.e. its thickness is remarkably smaller than its transverse dimension q. This is, for example, apparent from FIG. 4 that shows a cross-section of electrode 20 at the chain-dotted cutting line IV-IV in FIG. 3. The thickness d is smaller than ⅕, preferably smaller than 1/10 of the transverse dimension q.

As further apparent from FIG. 4, electrode 20 comprises two flat sides 23, 24 that are connected by means of narrow sides 25, 26. Thus, in total a quadrangular, preferably rectangular cross-section Q is obtained that is bordered by the flat sides 23, 24 and narrow sides 25, 26. The quadrangular cross-section can also be bent one time or multiple times, e.g. S-shaped.

Electrode 20 comprises a tapering section at its distal end in which the narrow sides 25, 26—extending parallel to one another apart therefrom—are convergingly arranged toward the tip 22. The convergingly toward one another extending sections of the narrow sides 25, 26 can be configured in a straight manner, as shown in FIG. 3, or also in a convex or concave manner. They define an angle α between each other that is preferably in the range of 20° to 100°.

As the cross-sections V-V and VI-VI show, that are separately illustrated in FIGS. 5 and 6, the cross-section of electrode 20 decreases toward tip 22 in distal direction D or in other words increases in proximal direction P. Thereby the thickness d of electrode 20 can be constant in the tapering section toward tip 22, as shown in FIGS. 5 and 6. The thickness d can, however, also decrease toward tip 22. However, in any case the transverse dimension q decreases in the tapering section toward tip 22.

In a first embodiment electrode 20 consists entirely of a material being well thermally conductive, as e.g. tungsten, a hard metal, copper, aluminum, or a combination of these materials. Thereby metals and also non-metallic electrically conductive materials can be used, such as DLC or a combination of metal and such materials. However, in any case the used material then has a thermal conductivity λ that is larger, preferably remarkably larger than the thermal conductivity of stainless steel. Particularly is λ≥50 W/(m*K), ≥100 W/(m*K), ≥200 W/(m*K), ≥300 W/(m*K), ≥400 W/(m*K).

In a preferred embodiment electrode 20 comprises a multiple layer configuration, as apparent from FIGS. 4 and 6. For this electrode 20 comprises an electrode base body 27 that is connected with a heat dissipation device 28 at least on its flat sides 23, 24, however as an option also on its narrow sides 25, 26. The heat dissipation device 28 consists in the present embodiment of a two-dimensional coating of the flat sides of the electrode base body 27 with thermally conductive overlays 29, 30. In the embodiment the base body 27 can consist of stainless steel, whereas the overlays 29, 30 consist of another material having a better thermal conductivity and/or a better electrical conductivity. Silver has shown to be particularly suitable for this purpose. Possible other overlays consist of aluminum and/or copper and/or hard metal and/or DLC and/or tungsten and/or a layer, e.g. metal layer with CBN (cubic boron nitride), diamond powder or similarly well thermally conductive material embedded therein.

The instrument described so far operates as follows:

As illustrated in FIG. 7, a gas stream 31 originating from the gas source 12 flows through lumen 19 of instrument 10 during operation. This gas stream (preferably argon stream) flows along both flat sides 23, 24 of electrode 20. Concurrently electrode 20 is supplied with radio frequency electrical current via wire 21. The working frequency of generator 13 and thus the frequency of the current is thereby preferably above 100 kHz, preferably above 300 kHz, further preferably above 500 kHz. At the tip 22 and an adjoining area thereof the current exits electrode 20 and forms a spark igniting toward the not further illustrated biological tissue of the patient or a plasma 32 flowing thereto. The root point 33 of the spark or plasma thereby touches the narrow sides 25, 26, however, particularly the flat sides 23, 24 of electrode 20, whereby this root point area 33 occupies at least 1/10 of the axial length (to be measured in proximal direction) of the area of electrode 20 in which the narrow sides 25, 26 diverge away from tip 22. The overlays 29, 30 extend into this area and preferably up to the tip 22. Thus, the spark or plasma stream is electrically directly supplied by overlay 29, 30. The thickness of overlays 29, 30 can be relatively small. It has shown that already coatings being 10 to 20 μm thick result in a substantial extension of the lifetime of electrode 20 and to a substantially reduced material removal therefrom. Preferably the thickness of the overlays—consisting e.g. of silver—has an amount of 20 μm, 30 μm or 50 μm, whereby in case of a thickness of the electrode of, e.g. 0.1 mm, a thermal resistance of more than 400 W/(m*K) is obtained. The electrode 20 consisting of the material combination stainless steel/silver thereby comprises a phenomenal lifetime.

In a modified embodiment it is also possible to let the electrode cross-section increase in proximal direction not continuously, different to the embodiments described above, but in a step-like manner, i.e. in one or multiple steps. Such an embodiment is illustrated in FIG. 8. However, this embodiment also realizes the inventive concept, which is why for the description of this electrode 20′ it is referred to the embodiment according to FIGS. 1-7. The already introduced reference numerals are used in the following, whereby they are provided with an apostrophe for the purpose of distinction. The description above accordingly applies apart from the following particularities for the embodiment according to FIG. 8.

The electrode 20′ comprises a tip 22′ that can be formed here by the pointed or blunt end of a wire-shaped electrode section. This wire-shaped electrode section 34 comprises a core 35 that forms the base body 27′ and for its part can be configured as thin cylinder pin. The core 35 is provided with an overlay 29′ that here—as appropriate in connection with an electrode holding platelet 36—forms the heat dissipation device 28′. The electrode section 34 can be welded, crimped or otherwise connected with the electrode holding platelet 36. A substance bond connection is preferred, because of the better heat transfer. The electrode holding section 36 can consist of stainless steel or another material that is provided with a thermally conductive coating, such as tungsten, copper, aluminum, DLC or the like or that is configured of thermally conductive material, such as tungsten, copper, aluminum, DLC or the like.

In operation of instrument 10 with an electrode 20′ according to FIG. 8, an electrical discharge and thus the forming spark or plasma stream origins first from tip 22 and then from at least a part of the wire-shaped electrode section 34. The electrically and thermally conductive coating 29′, that is preferably a silver coating, remarkably reduces the electrical resistance of electrode 20 or 20′. The radio frequency alternating current of generator 13 concentrates in the outer layers of electrode 20, 20′ and in this manner flows substantially through coatings 29, 30, 29′. Thereby ohmic losses at the electrode 20, 20′ are minimized and in addition the reduced amount of heat is dissipated substantially better away from the discharge root by the coating and is distributed such that it can be transferred to the gas stream extensively.

In the improved instrument 10 electrode 20, 20′ is provided with a heat dissipation device 28, 28′, such that the thermal resistance of electrode 20, 20′ measured in longitudinal direction (in distal or proximal direction) is preferably ≥300 W/(m*K). In a preferred embodiment the heat dissipation device 28, 28′ is formed by an overlay 29, 30, 29′ that comprises a higher electrical conductivity and also a higher thermal conductivity compared with the material of the electrode base body 27, 27′.

### REFERENCE SIGNS

10 instrument
11 apparatus
12 gas source
13 generator
14 line (for current)
15 line (for gas)
16 neutral electrode
17 distal end of instruments 10
18 hose
19 lumen
20 electrode
21 wire
22 tip
q transverse dimension of electrode 20
d thickness of electrode 20
23, 24 flat sides of electrode 20
25, 26 narrow sides of electrode 20
Q cross-section of electrode 20
α angle between sections of narrow sides 25, 26
D distal direction
P proximal direction
λ thermal conductivity
27 electrode base body
28 heat dissipation device
29, 30 coatings
31 das stream
32 plasma
33 root point
34 wire-shaped electrode section
35 core
36 electrode holding section

1. An electrode arrangement for an electrosurgical instrument for plasma coagulation, the electrode arrangement comprising:
an electrode that comprises a tip orientated in distal direction, the electrode having a cross-section that increases in proximal direction from the tip,
wherein the electrode comprises a material or a material combination having a thermal conductivity that is larger than 20 W/(m*K).

2. The electrode arrangement according to claim 1, wherein the electrode has a maximum transverse dimension and a radius of curvature at its tip that is smaller than 1/10 of the maximum transverse dimension.

3. The electrode arrangement according to claim 1, wherein the transverse dimension is configured to continuously increase in proximal direction starting from the tip.

4. The electrode arrangement according to claim 1, wherein the electrode comprises steplessly configured edges originating at the tip.

5. The electrode arrangement according to claim 1, wherein the electrode is configured as a platelet that comprises two flat sides that are connected with each other by means of narrow sides.

6. The electrode arrangement according to claim 1, wherein edges are formed between the narrow sides and the flat sides.

7. The electrode arrangement according to claim 1, wherein the electrode comprises a base body that comprises at least one surface on which a heat dissipation device is attached.

8. The electrode arrangement according to claim 7, wherein the heat dissipation device is a layer arranged on the base body.

9. The electrode arrangement according to claim 8, wherein the electrode comprises at least one flat side, and the layer is configured to cover the entire flat side.

10. The electrode arrangement according to claim 1, wherein the heat dissipation device comprises a thermally conductive material.

11. The electrode arrangement according to claim 7, wherein the heat dissipation device comprises a metallic material.

12. The electrode arrangement according to claim 7, wherein the heat dissipation device comprises a non-metallic material.

13. The electrode arrangement according to claim 7, wherein the heat dissipation device is configured and arranged to extend up to an area that is provided for direct contact with a spark originating from the electrode.

14. The electrode arrangement according to claim 7, wherein the heat dissipation device comprises an electrical conductivity that is larger than the electrical conductivity of the base body.

15. The electrode arrangement according to claim 7, wherein the heat dissipation device has a thermal conductivity that is larger than the thermal conductivity of the base body.
